(19) United States
(12) Patent Application Publication
(10) Pub. No.: US 2020/0121246 A1
(43) Pub. Date: Apr. 23, 2020

(54) SYSTEMS AND METHODS FOR ACCURATE AND STANDARDIZED DIAGNOSIS OF MEDICAL CONDITIONS

(71) Applicant: Integrative Diagnosis LLC, Newbury, MA (US)

(72) Inventor: William F. Brady, Boston, MA (US)

(73) Assignee: Integrative Diagnosis LLC, Newbury, MA (US)

(21) Appl. No.: 16/162,810

(22) Filed: Oct. 17, 2018

(51) Int. Cl. A61B 5/00; G16H 50/20

(52) U.S. Cl. CPC A61B 5/4538; G16H 10/20; G16H 50/20

(57) ABSTRACT

A method for standardizing diagnosis of medical conditions. The method includes defining first order data to collect for a patient having at least one symptom and providing a computing system for assigning a diagnosis of a medical condition from possible diagnoses corresponding to possible medical conditions. The computing system includes a memory system storing analysis modules that include tissue-pathology characteristics databases for the possible medical conditions corresponding to the possible diagnoses. The method includes receiving in the memory system the first order data for the patient, comparing with the computing system the first order data to the tissue-pathology characteristics databases, and assigning fit scores for each of the possible diagnoses based on the comparison between the first order data and the tissue-pathology characteristics databases. The method includes assigning the diagnosis of the medical condition for the patient based on the fit scores.

### FIELD

The present disclosure generally relates to systems and methods for accurate and standardized diagnosis of medical conditions, and more particularly for the diagnosis of musculoskeletal disorders.

### BACKGROUND

The Background and Summary are provided to introduce a foundation and selection of concepts that are further described below in the Detailed Description. The Background and Summary are not intended to identify key or essential features of the claimed subject matter, nor are they intended to be used as an aid in limiting the scope of the claimed subject matter.

Neck and back pain are the cause of an estimated $88 billion burden on the healthcare system in the United States. Under the systems and methods presently known within the field, patient complaints or symptoms are classified at only a rudimentary level before proceeding with treatment options. There is limited formal diagnosis of the underlying musculoskeletal disorder, rather, broad classification systems prevail including: non-specific low back pain, symptom based classification such as acute or chronic categories as a function of duration, then as a responder, non-responder, or other in response to treatments provided to alleviate these symptoms.

### SUMMARY

One embodiment of the present disclosure generally relates a standardized diagnosis system for diagnosing a medical condition. The system includes a computing system configured to receive first order data collected for a patient having at least one symptom and to assign a diagnosis of the medical condition for the patient. A processing system and a memory system are provided within the computing system, the memory system storing diagnosis logic executable by the processing system to assign the diagnosis of the medical condition from possible diagnoses corresponding to possible medical conditions. A plurality of analysis modules are within the memory system, each of the plurality of analysis modules comprising a database of tissue-pathology characteristics for the possible medical conditions corresponding to the possible diagnoses. The computing system is further configured to compare the first order data to the databases of tissue-pathology characteristics within the plurality of analysis modules and to assign fit scores for each of the possible diagnoses based on the comparison. The computing system is further configured to assign the diagnosis of the medical condition for the patient based on the fit scores.

Another embodiment generally relates to a method for standardizing diagnosis of medical conditions. The method includes defining first order data to collect for a patient having at least one symptom and providing a computing system for assigning a diagnosis of a medical condition from possible diagnoses corresponding to possible medical conditions. The computing system includes a memory system storing analysis modules that include tissue-pathology characteristics databases for the possible medical conditions corresponding to the possible diagnoses. The method includes receiving in the memory system the first order data for the patient, comparing with the computing system the first order data to the tissue-pathology characteristics databases, and assigning fit scores for each of the possible diagnoses based on the comparison between the first order data and the tissue-pathology characteristics databases. The method includes assigning the diagnosis of the medical condition for the patient based on the fit scores.

Another embodiment generally relates to a non-transitory computer-readable medium storing a program executable to perform steps to assign a diagnosis of a medical condition from possible diagnoses corresponding to possible medical conditions for a patient having at least one symptom. The program includes analysis modules that include tissue-pathology characteristics databases for the possible medical conditions corresponding to the possible diagnoses. The assignment of the diagnosis includes requesting first order data for the patient and receiving the first order data for the patient, comparing the first order data to each of the tissue-pathology characteristics databases, and assigning fit scores for each of the possible diagnoses based on the comparison between the first order data and each of the tissue-pathology characteristics databases. The steps further include assigning the diagnosis of the medical condition for the patient based on the fit scores.

Various other features, objects and advantages of the disclosure will be made apparent from the following description taken together with the drawings.

### BRIEF DESCRIPTION OF THE DRAWINGS

The drawings illustrate embodiments for carrying out the disclosure. The same numbers are used throughout the drawings to reference like features and like components. In the drawings:

FIG. 1 is a schematic view of an exemplary system for standardizing diagnosis of a medical condition according to the present disclosure;

FIG. 2 depicts an exemplary method by which the system of FIG. 1 provides for a standardized diagnosis of a medical condition;

FIG. 3 depicts an exemplary patient symptom map as incorporated into the exemplary method of FIG. 2;

FIG. 4 depicts exemplary comparisons of the patient symptom map from FIG. 3 within the method of FIG. 2; and

FIGS. 5-10 depict further comparisons using the diagnosis engine depicted in the system of FIG. 1 within the method of FIG. 2.

### DETAILED DISCLOSURE

This written description uses examples to disclose embodiments of the present disclosure and also to enable any person skilled in the art to practice or make and use the same. The patentable scope of the invention is defined by the claims and may include other examples that occur to those skilled in the art. Such other examples are intended to be within the scope of the claims if they have structural elements that do not differ from the literal language of the claims, or if they include equivalent structural elements with insubstantial differences from the literal language of the claims.

Musculoskeletal disorders comprise a major portion of overall medical conditions in the United States and abroad, requiring medical care by chiropractors, physical therapists, medical doctors, and others in the medical field. While neck and back pain in the United States constitutes an $88 billion annual expense for receiving such treatment, the present model provides for an underwhelming return in terms of patient outcome. This is due in large part to a system in which treatment is based on the training of the provider rather than a complete, accurate and specific diagnosis of the patient's condition.

Additionally, the present inventor has identified that the systems and methods presently known in the medical field, which provide for predominantly broad classification into acute or chronic conditions, result in treatment plans that are not based on the true root cause of the problem. Instead of assigning a diagnosis of an underlying musculoskeletal disorder, treatment is rendered based on the symptoms themselves, training of the provider or broad classification systems. Unlike diseases and the like that have regulated medication treatment regimens, the treatment of musculoskeletal disorders is essentially open. Through extensive research and experience, the present inventor has identified that treatment without proper diagnosis has been proven ineffective and costly, yet providers typically downplay or disregard all together the importance of proper diagnosis within musculoskeletal disorders.

Moreover, the reliance on the provider's personal education and experience, rather than any form of systematic and standardized diagnosis process, creates a substantial risk of missing important data due to limited time with the patient, poor recall, or infrequent exposure of a particular symptom, for example. Accordingly, the present inventor has identified that the systems and methods presently known in the art are woefully inadequate for the treatment of musculoskeletal disorders, and a new systematic and standardized system of diagnosis is needed. More specifically, a system and method is required that provides accurate and repeatable results based on a numeric system of scoring.

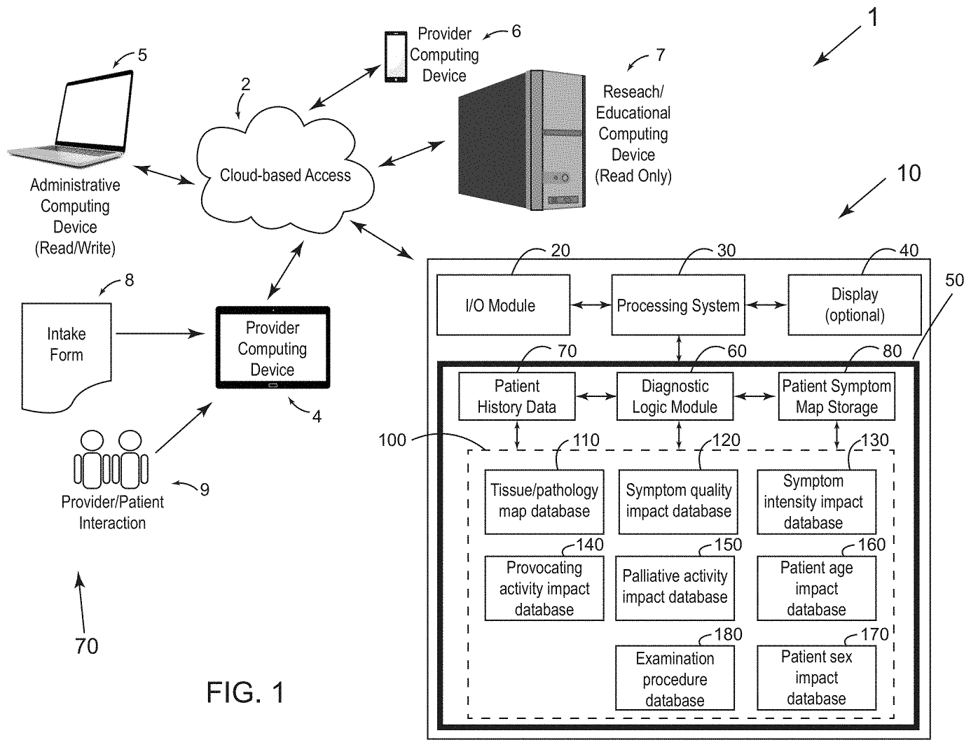

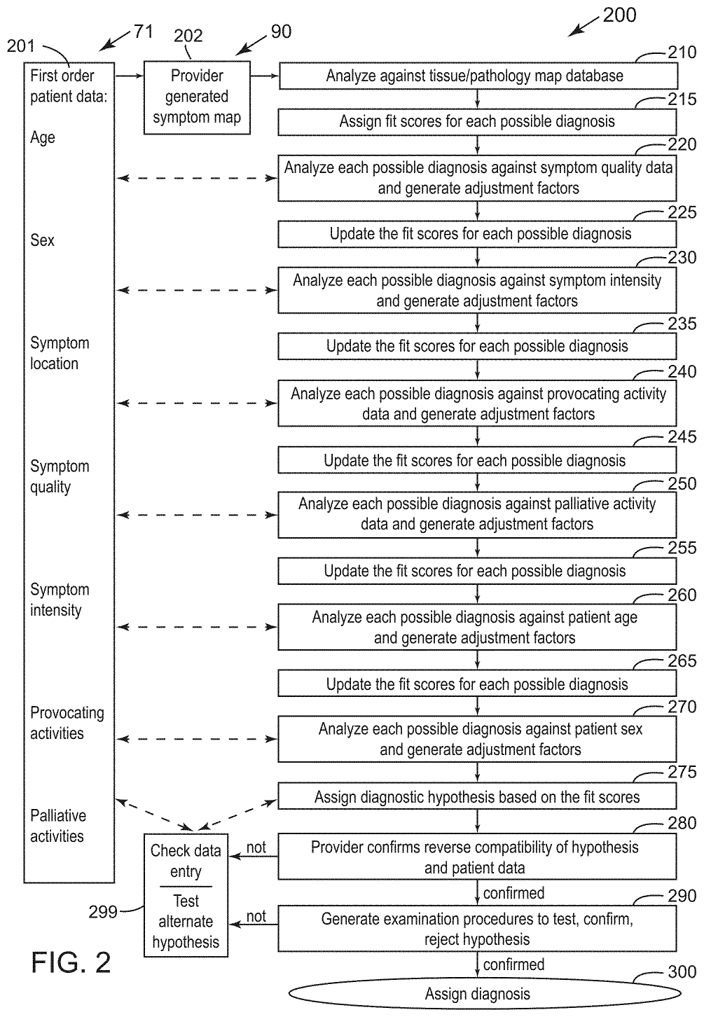

FIGS. 1 and 2 depict an exemplary system and method developed for the standardized diagnosis of a medical condition, including musculoskeletal disorders. Specifically, the presently disclosed systems and methods provide for a tissue and pathology specific diagnosis of the underlying musculoskeletal disorder. The system 1 of the embodiment shown comprises cloud-based access 2 between a provider using a provider computing device 4 and a computing system 10, which serves as a diagnosis engine. Using a centralized computing system 10 also enables different options for providers to subscribe to a service providing the presently disclosed systems and methods. For example, providers may pay a monthly or annual subscription fee for access to the computing system 10, such as a username and password via the cloud-based access 2 as is known in the art. Another option is to pay per patient entered into and analyzed by the computing system 10, including sub-options of storing patient data for subsequent reference or long-term use. Yet another use-based option is to pay based on the time that the provider spends using the computing system 10. Other options would also be recognized within this field and are also anticipated for controlled use and access to the systems and methods presently disclosed herein.

The provider computing device 4 is used to input the patient history data 70, shown here in the form of a patient intake form 8 and data collected from provider/patient interaction 9 (such as during an appointment), into the computing system 10, which ultimately analyzes the entirety of this patient history data 70 to provide a standardized diagnosis of the medical condition. The computing system 10 is configured to require a predetermined set of data as first order patient history data 71 within the patient history data 70. While these will be discussed further below, this first order patient history data 71 includes: patient age, patient sex, symptom location, symptom quality, symptom intensity, provocating activities, and palliative activities.

First order patient history data 71 is distinguished from second order patient history data (both forms shown stored together as patient history data 70 in FIG. 1), which relates to information such as how a problem came to be, rather than what the problem is. The systems and methods presently known in the art place substantial focus on this second order patient history data, which is often the bulk of the information provided by the patient. However, the present inventor has identified that the separation of data type, and explicitly limiting consideration of the data based on its type, are critical to an accurate diagnosis of the true underlying musculoskeletal disorder.

It should be recognized that although the system 1 is shown to have a single computing device 10 having cloud-based access 2 to the provider computing device 4 and others, the functions of the computing device 10 and/or provider computing device 4 may be combined or further separated. For the sake of clarity, the computing system 10 and processing system 30 are labeled as “systems” rather than implying a single device versus multiple devices to perform these functions. Other elements to be discussed further, such as the memory module or memory system 50, may also be divided across multiple modules within the same computing system 10, across multiple computing systems, and/or within a provider computing device 4.

In the embodiment shown, a computing system 10 is provided to enable provider computing devices 4 that would otherwise not have the necessary computing power to be used in the presently disclosed diagnosis system. This configuration further allows for one (or more) computing systems 10 to support many such provider computing devices 4. In the embodiment shown, the same cloud-based access 2 provides access to the provider computing device 4 as previously discussed, as well as others such as provider computing device 6, and also administrative computing devices 5, and/or research/educational computing devices 7.

Whether incorporated in the computing system 10, across several, or within the provider computing device 4, the computing system 10 presently shown incorporates an input/output module 20, a processing system 30, and an optional display 40. The input/output module 20 provides communication between the elements within the computing system 10 (such as the processing system 30) and devices connected to the cloud-based access 2 in a manner known in the art. It should also be recognized that while exemplary paths of connectivity between and within the elements of the computing system 10 are shown, alternate paths of communication are also anticipated by the present disclosure. Likewise, communication among and between elements of the computing system 10, whether within a single computing system 10 or across many, may be wired and/or wireless, using one or more technologies presently known in the art.

The computing system 10 further comprises a memory module 50 for storing data and executable programs, including the patient history data 70 previously discussed, as well diagnostic logic within the diagnostic logic module 16. The memory module 50 further includes a patient symptom map storage 80, as well as analysis modules 100 for processing data within the patient history data 70 in accordance with the diagnostic logic module 16 to assign a diagnosis of the medical condition, which is described further below.

Certain aspects of the present disclosure are described and depicted in terms of functional and/or logical block components and various processing steps. It should be recognized that any such functional and/or block components and processing steps may be realized by any number of hardware, software, and/or firmware components configured to perform the specified functions. For example, certain embodiments employ various integrated circuit components, such as memory elements, digital signal processing elements, logic elements, look-up tables, or the like, which are configured to carry out a variety of functions under the control of one or more processors or other control devices. The connections between functional and logical block components are also merely exemplary. Moreover, the present disclosure anticipates communication among and between such components being wired, wireless, and through different pathways.

These functions may also include the use of computer programs that include processor-executable instructions, which may be stored on a non-transitory tangible computer readable medium. The computer programs may also include stored data. Non-limiting examples of the non-transitory tangible computer readable medium are nonvolatile memory, magnetic storage, and optical storage. As used herein, the term module may refer to, be part of, or include an application-specific integrated circuit (ASIC), an electronic circuit, a combinational logic circuit, a field programmable gate array (FPGA), a processor system (shared, dedicated, or group) that executes code, or other suitable components that provide the described functionality, or a combination of some or all of the above, such as in a system-on-chip. The term module may include memory (shared, dedicated, or group) that stores code executed by the processor. The term code, as used herein, may include software, firmware, and/or microcode, and may refer to programs, routines, functions, classes, and/or objects. The term shared, as used above, means that some or all code from multiple modules may be executed using a single (shared) processor. In addition, some or all code to be executed by multiple different processors as a computer system may be stored by a single (shared) memory. The term group, as used above, means that some or all code comprising part of a single module may be executed using a group of processors. Likewise, some or all code comprising a single module may be stored using a group of memories as a memory system.

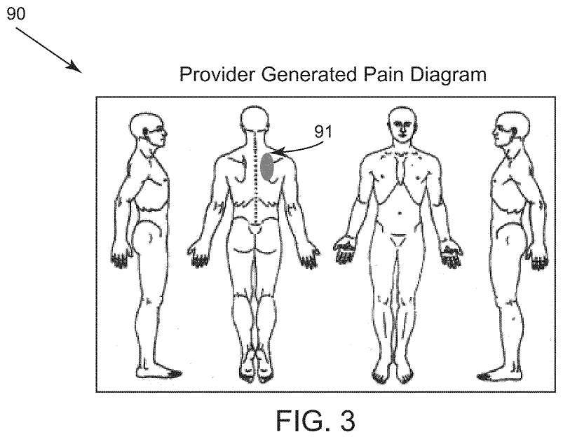

An exemplary process for using the system shown in FIG. 1 is provided in FIG. 2. The method 200 begins with the collection of pre-determined first order patient history data 71 in step 201, which as previously discussed is obtained through a patient intake form 8 as well as provider/patient interaction 9 during a medical consultation with the patient. In conjunction with the data collected in the patient intake form 8 and the provider/patient interaction 9, the provider further generates a patient symptom map 90 in step 202, which indicates the anatomy and specific location of the symptoms being experienced by the patient. This is unique relative to practices presently employed in the field, which require the patient to complete a pain diagram as part of the patient intake form 8. Even when considered in view of the patient's written descriptions in the patient intake form 8, the present inventor has identified that the results are very inaccurate and lack specificity. In contrast, the present inventor has identified that the patient symptom map 90 is highly accurate and effective in diagnosis when generated by the provider, which benefits from their formal training and experience with human anatomy and physiology. The provider may also ask questions to discern specific locations of pain based on anatomical landmarks in the body, such as being above or below the iliac crest, or 2″ posterior to the ASIS, for example. This data may be entered via mouse, keyboard, voice recognition, and/or other methods known in the art. An exemplary patient symptom map 90 is provided in FIG. 3, which demonstrates a pain location indicator 91 indicating where the provider identified the patient symptoms to reside. It should be recognized that in alternate embodiments, the patient symptom map 90 need not be a graphic, but may also include a selection of a specific anatomical location or landmark from a drop down list, for example. However, the present inventor has identified the selection of anatomy on a graphic to be particularly advantageous in producing accurate and consistent results.

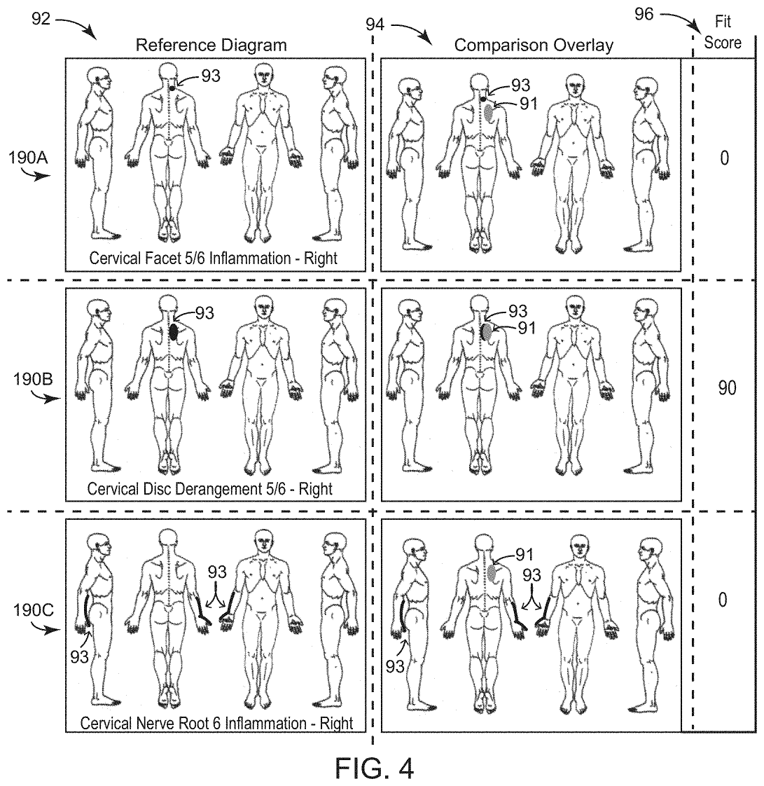

As shown in FIGS. 2-4, a patient symptom map 90 such as that shown in FIG. 3 is then compared in step 210 of the method 200 against a tissue/pathology map database 110 stored within the analysis module 100 of the computing system 10. FIG. 4 depicts exemplary reference maps 92 corresponding to three possible diagnoses, 190A-190C. It should be recognized that the content of the analysis modules 100 anticipate thousands of possible diagnoses corresponding to thousands of possible medical conditions to be diagnosed. However, a limited number of exemplary possible diagnoses 190A-190C are provided for the sake of clarity. In certain embodiments, some of the possible diagnoses will be removed entirely as consideration and/or display based on the patient history data 70 (i.e., female-only medical conditions for a male patient). In other embodiments, these possible diagnoses are not literally removed, but are placed at the bottom of a prioritized or sorted list of all possible diagnoses, for example.

Each reference map 92 includes a predetermined reference pain location indicator 93 in which a symptom is expected to reside for the medical condition associated with that possible diagnoses 190A-190C. Comparison overlay maps 94 are generated corresponding to the comparisons of each possible diagnoses 190A-190C, which shows the pain location indicator 91 from the patient symptom map 90 is compared to the reference pain location indicator 93 for each of the reference maps 92. Based on this comparison, a fit score 96 is assigned in step 215 for each of the possible diagnoses 190A-190C. In the embodiment shown, the second possible diagnosis 190B is assigned a fit score 96 of ninety based on the comparison between the pain location indicator 91 and the reference pain location indicator 93.

In certain embodiments, the fit score 96 is based on the proximity of the pain location indicator 91 and reference pain location indicator 93, the existence or number of indicators within a like region, alignment with known locations of referred pain, or criteria based on the particular possible diagnosis in question. In contrast, the method 200 provides that the particular differences in the exemplary locations between the pain location indicator 91 and the reference pain location indicator 93, whether based on distance or disassociation with the particular possible diagnoses 190A-190C, resulted in a score of zero for the first possible diagnosis 190A and the third possible diagnosis 190C. While the fit scores 96 of these possible diagnosis 190A-C may be updated or adjusted by other steps within the method, this first example provides that the second possible diagnosis 190B is the best fit or most likely explanation for the underlying medical condition to be diagnosed. By way of example, local tissues may include muscles, tendons, ligaments, cartilage, bone, nerve (cutaneous), or discs. Likewise, referred tissues may include scleratogenous versus nerve, and further root (dermatome) versus peripheral.

It will be readily apparent that this step alone is impossible under the systems and methods presently known, whereby the same tissue and/or pathology may be associated with literally hundreds of medical conditions. Moreover, although the same tissue and/or pathology may be associated with multiple medical conditions, the “fit” of each location with the underlying medical condition will be stronger in certain cases than in others, requiring further graduation in making these comparisons.

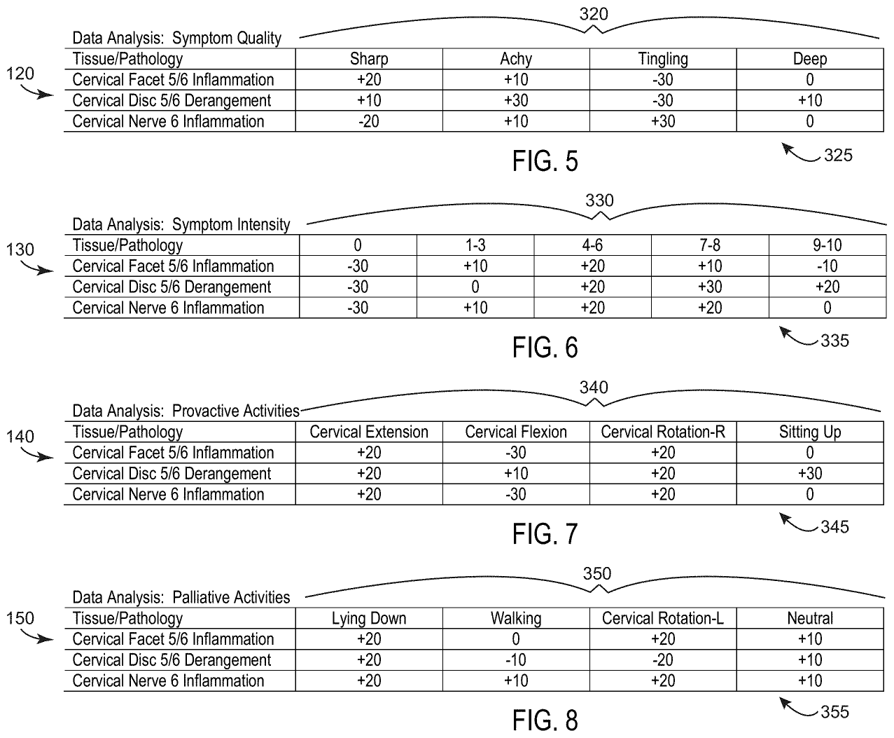

Returning to FIG. 2, the method 200 then analyzes each possible diagnoses 190A-190C against symptom quality data obtained within the first order patient history data 71 compared to data within the symptom quality impact database 120 of the analysis modules 100. As shown in FIG. 5, the results of this comparison are used to generate an adjustment factor in step 220, which is used to update the fit scores 96 for each of the possible diagnoses 190A-190C. It should be recognized that in certain cases, one or more of the fit scores 96 may not change following this analysis, or in other words, an adjustment factor may be positive values, negative values, or zero. FIG. 5 depicts four exemplary symptom quality types 320, as well as corresponding adjustment factors 325. Using the adjustment factors generated in step 220, the fit scores 96 for each possible diagnoses 190A-190C are updated accordingly in step 225.

It should be recognized that these adjustment factors 325 are merely exemplary, and greater or fewer symptom quality types 320 may be incorporated within the symptom quality impact database 120. By way of non-limited example, further symptom quality types 320 include: dull, numbness, burning, jolt, pins and needles, falling asleep, tearing, hot, cold, tender, swollen, blue, red, white, superficial, pinch, grabbing, and dozens of other provided descriptions. In certain embodiments, the present inventor has identified 30-50 categories to provide adequate selection options for the practitioner. While such data may be provided by the patient under the present systems and methods, there is currently no known comprehensive mechanism to interpreting this symptom quality data with respect to the possible diagnoses 190A-190C.

Next, the symptom intensity from the first order patient history data 71 is compared to data within the symptom intensity impact database 130 for each of the possible diagnoses 190A-190C within the analysis modules 100. In the embodiment of FIG. 6, the symptom intensity impact database 130 provides for five categories of symptom intensity ranges 330, which correspond to ranges of a scale of intensity or pain between zero and ten as is conventionally used in the field. Each of the range selections within the symptom intensity range 330 has a corresponding adjustment factor 335 for each of the possible diagnoses 190A-190C.

In certain embodiments, available symptom intensity ranges include mild, moderate, or severe categories for one or more of pathology, load, inflammation, odds of sensitization, and/or odds of psychosocial disorder. For example, a high symptom intensity may add psychosocial disorder as a diagnostic category available for selection, specifically prompting the provider to ask about stress, emotional triggers, life/work satisfaction, and the like. In this manner, the presently disclosed system and process provides for intelligent data gathering on a need to know basis. In further embodiments, the symptom intensity impact database 130 is used to generate a symptom intensity score itself, including based on the formula: symptom intensity=(pathology quantity)+(pathology severity)+(tissue load)+(inflammation)+(sensitization)+(psychosocial disorder), for example. However, it should be recognized that other equations and mechanisms for deriving symptom intensity scores are also anticipated by the present disclosure. Based on the selection of symptom intensity within the first order patient history data 71, updated fit scores are assigned for each possible diagnoses 190A-190C in step 235.

It should be recognized that analysis of the first order patient history data 71 within each of the analysis modules 100 need not occur within the order prescribed in the method 200 shown in FIG. 2. However, this analysis and corresponding updating of fit scores 96 in a serial, ordered manner was provided for clarity.

As shown in FIGS. 2 and 7, data corresponding to provocating activities collected in the first order patient history data 71 is then compared and analyzed against data within the provocating activity impact database 140 for each possible diagnoses 190A-190C. Provocating activity data generally relates to those activities that, when engaged in, increase the symptoms reported by the patient. Generally, these relate to three primary categories of loading: stretch, contract, and compress. Thousands of activities are stored and characterized within these categories, such as golfing, lifting a backpack or bag, or shrugging the shoulder. In other words, a database of activities is further divided into whether the activity causes loading in the form of stretch, contraction, or compression with respect to each tissue/pathology and thus each possible diagnoses 190A-190C. In this manner, the information that ascending stairs provokes symptoms will increase the likelihood of certain medical conditions being present, decrease the likelihood of others, and have no relevance or impact for others yet. In certain embodiments, symptom intensity data will also be collected with respect to the provocating activities to distinguish slight provocation from severe.

As with the modules from within the analysis modules 100 previously discussed, the exemplary provocating activity impact database 140 includes four provocating activity types 340, along with corresponding adjustment factors 345 for each of the possible diagnoses 190A-190C. The provocating activity types 340 presently listed are those most relevant to the current “best fits” for possible diagnoses 190A-190C, though others would also be stored within the provocating activity impact database 140. Based on the selections of activities that provoke symptoms in the patient as collected in the first order patient history data 71, the fit scores 96 are updated for each of the possible diagnoses 190A-190C in step 245.

Similarly to that previously discussed with provocating activities in step 240, step 250 of the method 200 provides for analyze palliative activity data from the first order patient history data 71 against data within the palliative activity impact database 150. To the extent this information is known and shared by the patient, the systems and methods presently known in the art lack any systematic, comprehensive or score value for the palliative data assessment. Most of the focus is on the symptom and when it started or what aggravates it at a highly-general level. FIG. 8 provides for four exemplary palliative activity types 350, which have corresponding adjustment factors 355 for each of the possible diagnoses 190A-190C. Once again, the palliative activity types 350 shown are those most relevant to the “best fits” for possible diagnoses 190A-190C; however, others would also be stored in the palliative activity impact database 150. As with provocating activities, the palliative activity impact database 150 stores thousands of activities, but this time with corresponding categories of unloading: approximation, decompression, and maintenance of resting or neutral state, each being specific to the activity and tissue/pathology. Examples of palliative activities include lying down, walking, traction, or not doing something, such as shrugs, golf, or lifting in a particular manner. Based upon the palliative activities provided by the patient in the first order patient history data 71, the fit scores 96 are updated for each of the possible diagnoses 190A-190C in step 255.

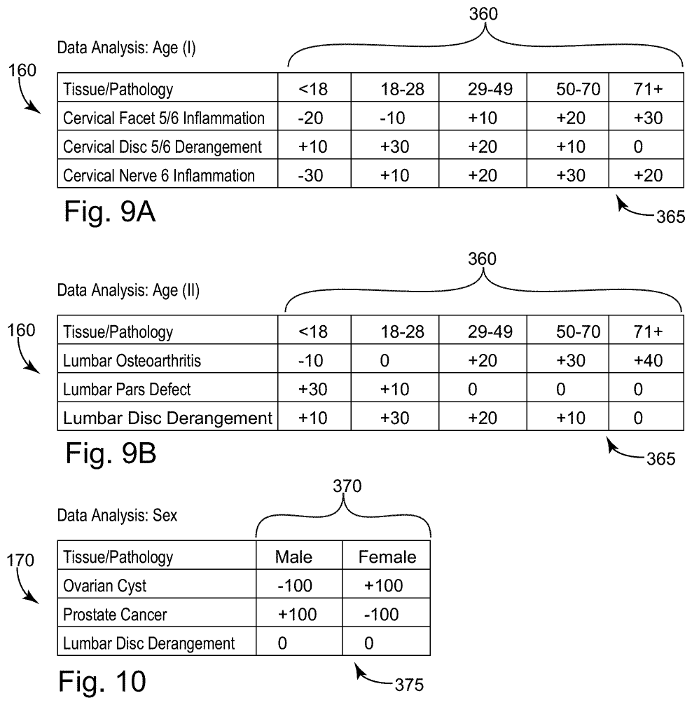

In the embodiment shown, the patient's age as provided in the first order patient history data 71 is then compared and analyzed with the data in the patient age impact database 160 within the analysis modules 100. Exemplary data within the patient age impact database 160, along with age ranges 360 and corresponding adjustment factors 365 are provided in FIGS. 9A and 9B. In certain embodiments, these age ranges 360 are provided for each decade of life. In other embodiments, the age ranges 360 do not cover a uniform set of years (such as shown in FIGS. 9A and 9B). In further embodiments, children have age ranges 360 encompassing fewer years so as to accommodate their more rapid growth, for example. In addition to the data shown in FIG. 9, further examples of adjustment factors provide that disc degeneration is much more likely with advanced age, whereas stress fractures only somewhat more likely with advanced age (perhaps until geriatric age). In general, the older the patient, the more pathology locations. Based on the patient age provided in the first order patient history data 71, updated fit scores 96 are provided for each of the possible diagnoses 190A-190C.

Next, the patient sex provided in the first order patient history data 71 is analyzed relative to the patient sex impact database 170. FIG. 10 shows two sex types 370 and corresponding adjustment factors 375. Based on the patient's selection of patient sex in the first order patient history data 71, updated scores for each possible diagnoses 190A-190C are provided. In certain cases, the selection of one sex will entirely eliminate the possibility of one of more of the possible diagnoses 190A-190C (i.e., update the score to zero, where it will remain). For example, a selection of “male” within the two sex types 370 will update the fit score 96 corresponding to an ovarian cyst as zero, as males do not have ovaries.

In the method 200 shown in FIG. 2, this step of updating the fit scores 96 is not explicitly shown since all first order patient history data 71 has now been analyzed with respect to all of the analysis modules 100. Instead, step 275 of the method 200 shows the assignment of a diagnostic hypothesis based on the final fit scores 96, or in other words based on the highest fit score 96 of the possible diagnoses 190A-190C. In the embodiment shown, the diagnostic hypothesis is the possible diagnosis 190A-190C having the highest score, though alternative “best fits” are also anticipated by the present disclosure, such as a low score, being closest to a 100% fit, or the like.

In the embodiment shown in FIG. 2, the diagnostic hypothesis assigned in step 275 (i.e., cervical facet ⅚ inflammation) must be confirmed by the provider to be reverse compatible in step 280, as compared with the provider's hypothesis and the patient history data 70 provided. In other words, step 280 requires the provider to confirm that the diagnostic hypothesis assigned in step 275 makes sense before it is officially determined to be the diagnosis of the patient's medical condition. If the provider confirms this diagnostic hypothesis is not incompatible, the method 200 may optionally provide for additional examination procedures to further test, confirm, or reject the diagnostic hypothesis assigned in step 275. This is shown as step 290, which is based on data from the examination procedure database 180 within the analysis modules 100. For example, the provider may be instructed or suggested to put the patient in one or more conditions of loading or unloading to add further data regarding provocating activities, palliative activities, symptom quality, and/or symptom intensity. As another example, the provider may be instructed to palpate or otherwise feel for certain conditions that would increase or decrease the likelihood of a particular medical condition and thus, its corresponding diagnoses.

If instead the provider does not confirm that diagnostic hypothesis to be reverse compatible with the first order patient history data 71, the process continues by checking the data entry of the first order patient history data 71 and patient symptom map 90, or the testing of an alternative hypothesis. For example, the provider may proceed with testing the second-highest fit score 96 among the possible diagnoses 190A-190C.

In the event that examination procedures are generated and proposed in step 290, and that these further examination procedures either reject the existing diagnostic hypothesis or further support another to the extent that the other becomes the “best fit,” the process continues with either checking the data entry of the first order patient history data 71 or the patient symptom map 90, or testing an alternative hypothesis in step 299. If instead no further examination procedures are generated in step 290, or the results of such examination procedures confirm (or simply do not reject) the diagnostic hypothesis assigned from step 275, the diagnostic hypothesis is assigned as a diagnosis of a medical condition for the patient in step 300.

From there, the diagnosis assigned in step 300 may be displayed on the provider computing device 4 or otherwise recorded. In certain embodiments, treatment options are also provided by the computing system 10 that correspond with the diagnosis assigned in step 300. These treatment options may then be considered by the provider to be used in addition, or in place of the provider's prior concepts for treatment of the properly-assigned diagnosis.

In certain embodiments, patient response to treatment is further inputted into the system 1 as a measure of changes in the patient's symptoms. This may be incorporated into further diagnosis of additional or alternate medical conditions if symptoms persist or new symptoms arise. In further embodiments, this information is further incorporated into machine learning within the computing system 10 to further improve the accuracy of the method 200, improve treatment outcome and/or timing, and/or to accommodate new knowledge within the field with respect to medical conditions or treatment options thereof.

As discussed above, the present inventor has identified that a critical component of the presently disclosed systems and methods is the explicit selection of seven items within the first order patient history data 71, age, sex, symptom location, symptom quality, symptom intensity, provocating activities, and palliative activities, to ensure optimal diagnosis power and accuracy. In particular, the present inventor has identified that omitting any of these seven particular types of first order patient history data 71 result in a poor fit score 96 and poor diagnosis reliability for missing critical data.

In contrast, the present inventor has also identified that among the greatest problems in the ad-hoc systems presently known in the art is that too much data is collected and considered. In particular, these superfluous additional pieces consume precious time to both collect and analyze this data, as well as complicate and obfuscate the analysis process—all of which lead to inferior diagnostic results. For example, in the case in which a patient has recently been involved in an automobile accident, the provider/patient interaction generally includes such items as who else was in the car, what time of day it was, and other background information regarding the order of events leading up to the accident. This is a misuse of provider time and attention, and also distracts from obtaining tissue and pathology specific diagnostic information.

In certain embodiments, information above and beyond the seven types collected as first order patient history data 71 are classified as second order patient history data within the overall storage of patient history data 70. In certain embodiments, the second order patient history data is not involved in the preliminary assignment of a diagnostic hypothesis in step 275 for the reasons provided above. However, this second order patient history data (which may also include historic diagnosis of the patient by the system 1) may be incorporated into the provider's confirmation of reverse compatibility in step 280 in determining the final diagnosis assigned in step 300.

In addition to providing the accuracy and reproducibility of a method 200 incorporating numeric scoring, the presently disclosed systems and methods provide unprecedented consideration of millions of considerations, many of which having interdependencies. Likewise, the presently disclosed systems and methods ensure consistency across providers and even provider types (i.e., chiropractor, PT, MD) and also providing an alternative to the steep learning curve of conventional methods in rendering care to a patient.

In the above description, certain terms have been used for brevity, clarity, and understanding. No unnecessary limitations are to be inferred therefrom beyond the requirement of the prior art because such terms are used for descriptive purposes and are intended to be broadly construed. The different assemblies described herein may be used alone or in combination with other devices. It is to be expected that various equivalents, alternatives and modifications are possible within the scope of any appended claims.

I claim:

1. A standardized diagnosis system for diagnosing a medical condition, the system comprising:
a computing system configured to receive first order data collected for a patient having at least one symptom and to assign a diagnosis of the medical condition for the patient;
a processing system and a memory system within the computing system, the memory system storing diagnosis logic executable by the processing system to assign the diagnosis of the medical condition from possible diagnoses corresponding to possible medical conditions;
a plurality of analysis modules within the memory system, each of the plurality of analysis modules comprising a database of tissue-pathology characteristics for the possible medical conditions corresponding to the possible diagnoses;
wherein the computing system is further configured to compare the first order data to the databases of tissue-pathology characteristics within the plurality of analysis modules and to assign fit scores for each of the possible diagnoses based on the comparison; and
wherein the computing system is further configured to assign the diagnosis of the medical condition for the patient based on the fit scores.

2. The standardized diagnosis system according to claim 1, wherein in assigning the diagnosis, the computing system is further configured to assign a diagnostic hypothesis of the medical condition for the patient based on the fit scores, to test the diagnostic hypothesis to confirm reverse compatibility with the first order data, and to assign the diagnosis only when the diagnostic hypothesis is confirmed to be reverse compatible.

3. The standardized diagnosis system according to claim 2, wherein the computing system is further configured to receive second order data for the patient, and wherein testing the diagnostic hypothesis further includes confirming reverse compatibility with the second order data.

4. The standardized diagnosis system according to claim 1, wherein in assigning the diagnosis, the computing system is further configured to assign a diagnostic hypothesis of the medical condition for the patient based on the fit scores, to generate an examination procedure to test the diagnostic hypothesis, and to assign the diagnosis when the examination procedure confirms the diagnostic hypothesis.

5. The standardized diagnosis system according to claim 1, wherein the computing system is further configured to receive a patient symptom map identifying a location of the at least one symptom on the patient, wherein one of the databases within the plurality of analysis modules comprises a tissue-pathology map database, and wherein the computing system is configured to compare the patient symptom map with the tissue-pathology map database and to update the fit scores for each of the possible diagnoses based on the comparison between the patient symptom map and the tissue-pathology map database.

6. The standardized diagnosis system according to claim 1, wherein the first order data comprises seven categories of data, and wherein the seven categories include age, sex, symptom location, symptom quality, symptom intensity, provocating activities, and palliative activities.

7. The standardized diagnosis system according to claim 1, wherein the computing system is further configured to generate a treatment recommendation based on the diagnosis assigned.

8. The standardized diagnosis system according to claim 7, wherein the computing system is further configured to receive follow-up data relating to an effectiveness of the treatment recommendation in treating the medical condition, and to incorporate the effectiveness into the computing system for future assignment of diagnoses.

9. The standardization diagnosis system according to claim 1, wherein the computing system is a central server accessible by a plurality of remote devices, and wherein the first order data is first received with one of the plurality of remote devices and sent to the computing system.

10. A method for standardizing diagnosis of medical conditions, the method comprising:
defining first order data to collect for a patient having at least one symptom;
providing a computing system for assigning a diagnosis of a medical condition from possible diagnoses corresponding to possible medical conditions, the computing system comprising a memory system storing analysis modules that include tissue-pathology characteristics databases for the possible medical conditions corresponding to the possible diagnoses;
receiving in the memory system the first order data for the patient;
comparing with the computing system the first order data to the tissue-pathology characteristics databases;
assigning fit scores for each of the possible diagnoses based on the comparison between the first order data and the tissue-pathology characteristics databases; and
assigning the diagnosis of the medical condition for the patient based on the fit scores.

11. The method according to claim 10, wherein the step of assigning the diagnosis includes assigning a diagnostic hypothesis of the medical condition for the patient based on the fit scores, testing the diagnostic hypothesis to confirm reverse compatibility with the first order data, and assigning the diagnosis only when the diagnostic hypothesis is confirmed to be reverse compatible.

12. The method according to claim 11, further comprising receiving second order data for the patient, wherein the step of testing the diagnostic hypothesis further includes confirming reverse compatibility with the second order data.

13. The method according to claim 10, wherein the step of assigning the diagnosis includes assigning a diagnostic hypothesis of the medical condition for the patient based on the fit scores, generating an examination procedure to test the diagnostic hypothesis, and assigning the diagnosis when the only when the examination procedure confirms the diagnostic hypothesis.

14. The method according to claim 10, wherein the databases include a tissue-pathology map database, further comprising receiving in the memory system a patient symptom map identifying a location of the at least one symptom on the patient, comparing with the computing system the patient symptom map relative to the tissue-pathology map database, and updating the fit scores for each of the possible diagnoses based on the comparison between the patient symptom map and the tissue-pathology map database.

15. The method according to claim 10, wherein the first order data comprises seven categories of data, and wherein the seven categories include age, sex, symptom location, symptom quality, symptom intensity, provocating activities, and palliative activities.

16. The method according to claim 10, wherein the first order data is received through both a patient intake form and through interaction between the patient and a medical provider.

17. The method according to claim 10, further comprising generating a treatment recommendation based on the diagnosis assigned.

18. The method according to claim 17, further comprising receiving follow-up data relating to an effectiveness of the treatment recommendation in treating the medical condition, and incorporating the effectiveness into the computing engine for future assignment of diagnoses.

19. A non-transitory computer-readable medium storing a program executable to perform steps to assign a diagnosis of a medical condition from possible diagnoses corresponding to possible medical conditions for a patient having at least one symptom, the program comprising analysis modules that include tissue-pathology characteristics databases for the possible medical conditions corresponding to the possible diagnoses, the assignment of the diagnosis comprising:
requesting first order data for the patient and receiving the first order data for the patient;
comparing the first order data to each of the tissue-pathology characteristics databases;
assigning fit scores for each of the possible diagnoses based on the comparison between the first order data and each of the tissue-pathology characteristics databases; and
assigning the diagnosis of the medical condition for the patient based on the fit scores.

20. The non-transitory computer-readable medium according to claim 19, wherein the first order data is received through both a patient intake form and through interaction between the patient and a medical provider, wherein the first order data comprises seven categories that include age, sex, symptom location, symptom quality, symptom intensity, provocating activities, and palliative activities, wherein the databases include a tissue-pathology map database, further comprising performing the step of receiving a patient symptom map identifying a location of the at least one symptom on the patient, comparing the patient symptom map to the tissue-pathology map database, and updating the fit scores for each of the possible diagnoses represented within the tissue-pathology map database; and wherein assigning the diagnosis includes assigning a diagnostic hypothesis of the medical condition for the patient based on the updated fit scores, testing the diagnostic hypothesis to confirm reverse compatibility with the first order data, and assigning the diagnosis only when the diagnostic hypothesis is confirmed to be reverse compatible.
